(19) United States
(12) Patent Application Publication
(10) Pub. No.: US 2010/0228071 A1
(43) Pub. Date: Sep. 9, 2010

(54) Adsorbents for Purification of C2-C3 Olefins

(75) Inventors: Prakash Kumar, Gujarat (IN); Ravi Puranik Vijayalaxmi, Gujarat (IN); Pavagada Raghavendra Char, Gujarat (IN); Sodankoor Garadi Thirumaleshwara Bhat, Gujarat (IN)

Correspondence Address: LADAS & PARRY LLP, 26 WEST 61ST STREET, NEW YORK, NY, 10023, US

(73) Assignee: INDIAN PETROCHEMICAL CORPORATION LIMITED, Gujarat (IN)

(21) Appl. No.: 11/990,298

(22) PCT Filed: Nov. 10, 2005
(86) PCT No.: PCT/IN2005/000365

§ 371 Date: Apr. 1, 2010

(30) Foreign Application Priority Data
Aug. 9, 2005 (IN) 919/MUM/2005

(51) Int. Cl. C07C 7/12; B01J 29/06

(52) U.S. Cl. 585/830; 502/64; 502/62; 585/820

(57) ABSTRACT

An adsorbent for removing impurities such as CO₂, H₂S and water vapors from a gaseous olefin stream of at least C₂ to C₄ olefins is disclosed. The adsorbent comprises of zeolite CaA molecular sieve modified with metal silicate.

### FIELD OF INVENTION

The present invention relates to use of adsorbents in purification of impure C₂-C₃ olefins such as typically produced in polymerization of olefins and produced as off gas. More particularly, the present invention purification of C₂-C₃ olefins by passing an impure C₂-C₃ olefinic stream containing low concentration carbon dioxide as impurity along with methane and ethane gases over an zeolite molecular sieve adsorbent bed by using Temperature Swing Adsorption process (TSA). The present invention also relates to a method of preparation of the adsorbent.

### BACKGROUND OF THE INVENTION

Light olefins (C₂-C₃) serve as building blocks for the production of numerous chemicals. C₂-C₃ olefins have traditionally been produced through the process of steam or catalytic cracking. Ethylene or propylene, the light olefins have a great number of commercial applications particularly in the manufacture of polyethylene, polypropylene, isopropyl alcohol, ethylene oxide, ethylene glycol etc. When polyethylene or polypropylene are manufactured monomers like propylene, ethylene, catalysts, and solvents are contacted at pressure in a reactor to produce polyethylene and polypropylene. The raw polymer product is produced in powder form and contains significant quantities of unreacted monomers and other raw materials. These unreacted monomers are constantly removed from the powder to avoid buildup of the low concentration impurities like carbondioxide, ethane, moisture etc., to generate off gas containing predominantly high value C₂-C₃ monomer, which quite often is sent to flare or used as fuel because of low concentration impurities. Polymer plants in petrochemical units have to eliminate carbon dioxide, which is well known catalyst inhibitor in monomers such as ethylene, propylene, butadiene, etc., to prevent poisoning of the polymerization catalysts and deterioration of polymer properties.

### SUMMARY OF THE INVENTION

The present invention provides a method for removing carbon dioxide from olefinic gaseous streams of polyolefin plant off gases and is particularly effective for removing low concentration of carbon dioxide. The requirement of CO₂ removal are very stringent (down up to 1 ppm) in the gaseous olefin streams and is most difficult to remove from low molecular weight olefins such as ethylene and propylene. Several methods are known for purification of olefinic streams like cryogenic distillation, liquid absorption, membrane separation and pressure swing adsorption.

Various options are being practiced in industry like caustic or mono ethanaloamine (MEA) scrubbers for CO₂ removal from a gaseous streams but have the disadvantages of being hazardous, non regenerable with continuous addition of scrubbing solution to the stream which renders it an on lucrative option. Regenerable chemisorption based solid amine sorbents are disclosed by Birbara et al in U.S. Pat. No. 5,876,488 to remove CO₂ from gaseous streams. Another approach has been to use base containing alumina adsorbents employing chemisorption or reversible chemical reactions to bind carbon dioxide to the metal carbonates or bicarbonates (U.S. Pat. No. 4,433,981, Slaugh, U.S. Pat. No. 3,865,924 Gidaspow). Main disadvantage of these reversible chemisorption adsorbents is low operational reliability, short life due to the tendency of active components to sinter and low ppm level CO₂ capacity. Temperature swing adsorption process using adsorbents like base containing alumina and zeolite molecular sieves are quite often used for purification of olefinic streams.

Preferred zeolite molecular sieves include commercially available sieves for CO₂ adsorption for example are zeolite A, zeolite X, zeolite Y, zeolite ZSM, mordenite, and their mixtures. The cations present in these zeolites include Na⁺, Ca²⁺, Mg²⁺ and combinations thereof. Silicon to aluminum ratio varied in the range of 1 to 5. A number of patents disclose molecular sieve adsorbents having improved adsorption capacities, especially for the removal of carbon dioxide from gas mixture. For example, U.S. Pat. No. 2,882,244, Milton discloses a variety of crystalline alumino silicates useful for CO₂ adsorption. In US Patent 3078639, Milton discloses zeolite X useful for adsorption of carbon dioxide from gas stream comprising of ethylene. In U.S. Pat. No. 6,530,975 Rode discloses the improvement of carbon dioxide adsorption capacity at very low partial pressures for purification of gaseous streams containing carbon dioxide and water vapors. Zeolite CaA molecular sieve has been used to co-adsorb CO₂ and H₂O from ethylene gas used for production of polyethylene at high pressure of 430, psig as detailed in “Gas Purification” chapter 12 “Gas Dehydration and Purification by Adsorption” page number 1076.

### SUMMARY OF THE INVENTION

Therefore, it is an object of the present invention to provide a process and adsorbent for the removal of low concentration CO₂ from olefinic gaseous streams employing a regenerable zeolite molecular sieve CaA adsorbent with enhanced CO₂ adsorption rate compared to olefin to remove carbon dioxide up to 1% from C₂-C₃ olefinic streams. Zeolite molecular sieve CaA and NaX are physical sorption based sorbents and have high equilibrium adsorption capacity for carbon dioxide, but CO₂ sorption capacity reduces to less than 1% in the presence of C₂-C₃ olefins because of co-adsorption of ethylene necessitating high volume of adsorbent, which is not a suitable option in polyolefin industry. The method comprises contacting the gaseous stream with an ZMS CaA prepared by modification with inorganic and organic silicates and drying and calcining the resultant, material at a temperature ranging from about 150 to 600° C., preferably 350 to 550° C. After use, heating to 120-250° C. in the presence of nitrogen can readily regenerate the adsorbent material. The prepared adsorbent is solid, stable, relatively non toxic which can be regenerated continuously using only heat or hot gases without deterioration with time. It can be used in packed beds and provides little or no dusting or carryover of fines. The rate at which the olefin stream is fed to the adsorbent bed is not critical but will vary with the reactor size but in any event, it should be a rate sufficient to effect efficient contact between feed and modified ZMS CaA adsorbent. This invention is well suited for continuous process in which olefin feed is continuously fed over a bed of modified ZMS CaA at the desired process conditions.

Therefore, high carbon dioxide dynamic capacity at very low partial pressures for C₂-C₃ olefins purification is the most important and required property of the adsorbent to treat polyolefin off-gases having typical composition like below.

### Typical Polyolefin Off Gas

| | |
|---|---|
| C1 ppm | 25-40 |
| C2% | 0.5-1 |
| C2% | Balance |
| C2 ppm | <1 |
| CO ppm | 0.2-0.5 |
| CO2% | 0.1-1 |
| Moisture ppm | <5-10 |
| OXYGEN ppm | <3 |
| Temp, C. | 35 |
| Flow, Kg/h | 2000 |
| Pressure, bar | 10-15 |
| Partial Pressure, bar | 0.074 (55.5 mmHg) CO2 |

### BRIEF DESCRIPTION OF THE ACCOMPANYING DRAWINGS

FIG. 1: CO2 fractional uptakes on zeolite A and modified samples at 30 C and 100 mmHg pressure.

FIG. 2: Ethylene fractional uptakes on zeolite A and modified samples at 30 C and 100 mmHg pressure.

FIG. 3: Carbon dioxide adsorption breakthrough's at 10.5 kg/cm2 pressure on various Zeolite Molecular sieve and modified samples.

FIG. 4: Schematic diagram showing adsorption breakthrough apparatus.

### DETAILED DESCRIPTION OF THE INVENTION

The zeolite molecular sieve (ZMS) adsorbents of this invention are prepared by coating the inorganic or organic silicate solution over the commercial version of the ZMS in extrudates or beads form. Inorganic silicates were prepared by mixing in the distilled water. Many inorganic silicates, sodium, potassium, calcium and lithium can be taken as coating material. Sodium and potassium silicates can be taken preferred material for coating of the zeolite molecular sieves to improve the diffusional uptake of the carbon dioxide in the presence of ethylene.

In the process for the modification of the calcium form of zeolite A, 1.5 mm to 3 mm extrudates of the adsorbent according to the invention are formed by, a) wetting the zeolite CaA with distilled water thoroughly, b) preparing the solution of inorganic silicate dissolved in suitable solvent like water in concentration range of 1 to 20%, c) coating by mixing the prepared silicate solution with zeolite molecular sieve with predetermined quantity of silicate solution in the range of 0.1 wt % to 15 wt % and equilibrated for a period or 0.1 to 24 hrs preferably, for 1 to 2hrs. d) removing excess prepared metal silicate solution from the resultant mixture by decanting. e) loading the adsorbent loaded in stainless steel ray in 1-2 cm thick layer and quick dried in oven at 100-200° C. with or without inert flow. f) the dried adsorbent is then calcined at a temperature in the range of 100-600° C. for a period of time from about 0.1 to about 100 hrs, preferably from about 1 to 10 hours. The heating step can be conducted in a suitable atmosphere such as nitrogen and helium. The calcium form of zeolite A (ZMS 5A) thus modified by inorganic silicates is named as PE5A in subsequent text.

Representative examples of the inorganic silicates that can be suitably used include, potassium silicate, sodium silicate and calcium silicate. Zeolite molecular sieve used for present invention can be in beads or extrudates form.

The adsorbent of the present invention can also be prepared by coating organosilicates over the ZMS X or calcium form of A type in extrudates or bead form. The organosilicate coating was achieved by a) preparation of organosilicate solution by dissolving in suitable organic solvent like toluene or acetone in the concentration range of 0.1 to 20%. b) previously activated ZMS A in the temperature range of 200-300° C. for 1 to 20 hrs is mixed with organosilicate solution to have homogeneous coating. c) excess of solvent is distilled off in the temperature range of 50 to 150° C. d) prepared dried adsorbent is calcined in temperature range of 90 to 650° C. preferably at, 400 to 550° C. for a period of time from about 0.1 to about 100 hrs, preferably from about 1 to 10 hours. The heating step can be conducted in a suitable atmosphere such as nitrogen and helium. The calcium form of zeolite A (ZMS CaA) thus modified with organo silicates is named as PET5A in subsequent text.

Representative examples of the organo silicates that can be suitably used include, tetraethyl silicate, tetra propyl silicate, tetrabutyl silicate and solvents for example, toluene, acetone, benzene and ortho-meta and paraxylenes, ZMS can be in either X or A form.

The absorbent of the present invention can also be prepared by ion exchanging the calcium form of zeolite A extrudates with inorganic or organic silicate solution prepared in the concentration range of 1-20% and solid to liquid ratio of ¼ and at the temperature of 60-90° C. The resultant solid mixture is heated at a temperature in the range of 90 to 650° C., preferably at 400 to 550° C. for a period of time from about 0.1 to about 100 hrs, preferably, from about 1 to 10 hours. The heating step can be conducted in a suitable atmosphere such as nitrogen and helium.

The adsorbents of this invention described above can be used to remove 0.01 to 2%, more specifically 0.01 to 1%, carbon dioxide from C₂-C₃ olefinic streams of polyolefin plant off-gases in petrochemical industry. The C₂-C₃ purification process comprises passing a stream of mixed gas through an adsorber bed charged with adsorbent. Adsorbent bed can be regenerated by heating with inert gas medium like nitrogen or helium at 100° to 220° C. or preferably, at 120-160° C. The adsorbent so regenerated can be reused as an adsorbent for carbon dioxide removal from ethylene or propylene gas. Purification process can also purify C₂-C₃ gases with higher concentration of carbon dioxide up to 15%.

The invention will now be further illustrated by the following examples. The adsorption rates are obtained by measuring carbon dioxide and ethylene adsorption capacity gravimetrically in a McBain balance. Water adsorption isotherms were measured gravimetrically. In a typical adsorption kinetics—measurement, a known quantity of the adsorbent was loaded in McBain balance and activated under vacuum (to 10⁻⁴ mmHg) at a suitable temperature for several hours. The adsorbent was then cooled to room temperature under vacuum. Adsorption uptakes were measured gravimetrically with pulse of pure gas into the adsorption set-up and fractional uptakes were calculated from the datum on amount of gas adsorbed in a given time on adsorbent. After each adsorption measurement, desorption experiment was also carried out to check the reversibility of the adsorption rates.

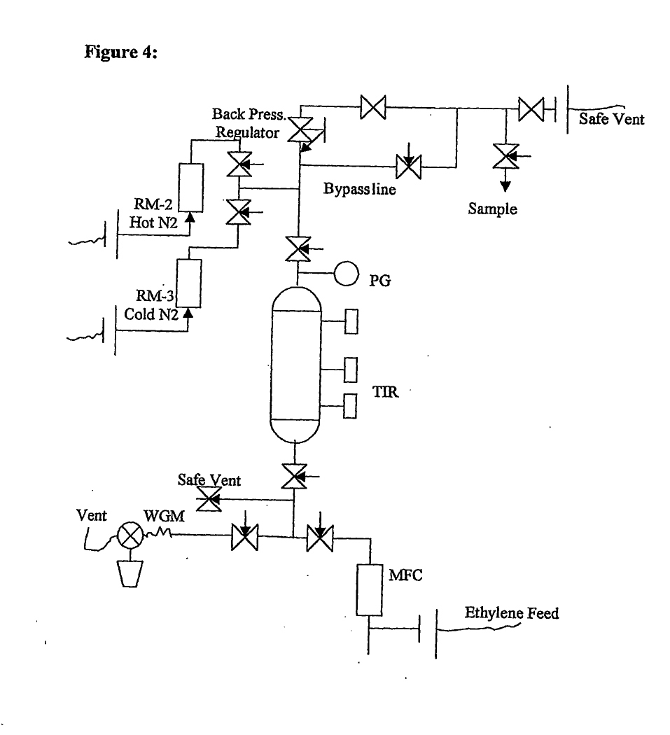

Further gas mixture adsorption breakthrough's were measured to estimate dynamic capacity at 30 to 80° C. and 10-20 Kg/cm² containing 0.01 to 1% of CO₂ balance ethylene, were measured on untreated sodium form, calcium form of ZMS A, pore modified calcium form of ZMS A and untreated zeolite NaX. Adsorption breathrough setup was comprised of 1″ internal diameter 50 cm long SS pipe. Five thermocouples were connected at different intervals to measure adsorbent bed temperature at different heights in the bed as shown in FIG. 4. Feed gas flow was controlled at inlet of bed by mass flow controller and a pressure gauge fixed at the top of the bed to measure bed pressure. Pressure in the bed was maintained by a back pressure regulator attached at the top of the bed. Flow of regeneration gas was controlled by a needle valve. Three tubular heaters were installed for heating adsorbent bed during regeneration and a three way valve attached at the bottom of the bed for venting out hot regeneration gas. Volume of the product and regeneration gas were measured by wet gas meters installed after the gas sampling points. Feed gas mixture containing 0.01 to 1 wt % carbon dioxide gas was prepared by mixing CO₂ and ethylene in gas cylinder. Analysis of feed gas, effluent regeneration gas, and product gas was done by GC method using a porapack Q column and TCD detector.

In order to illustrate the present invention and the advantages thereof, the following examples are provided. It is understood that these examples are illustrative and do not provide any limitation on the invention in the manner in which it can be practiced.

### Example 1

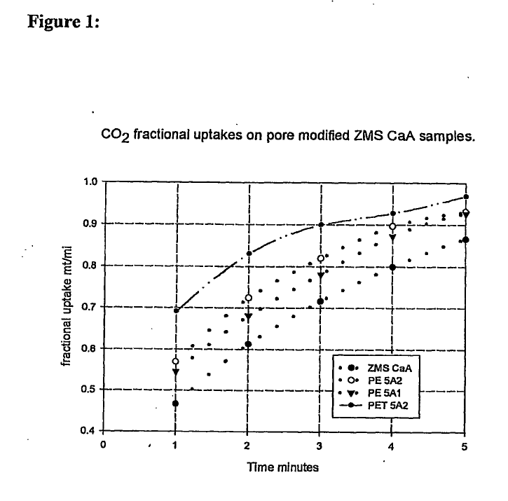

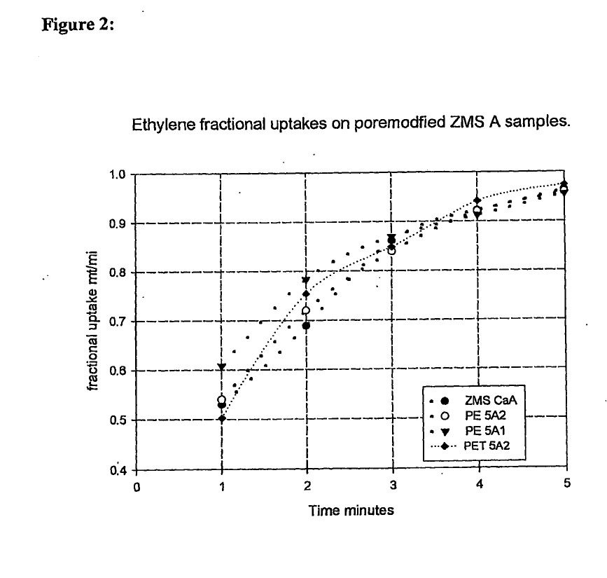

230 gm of 5A zeolite molecular sieve 1.5 mm extrudates were saturated with double distilled water and excess water decanted. 7.5 gm of metal silicate comprised of potassium dissolved in 200 gm of double distilled water to prepare 1% metal silicate solution (27 wt % metal silicate purity). The prepared solution was thoroughly mixed with water-saturated adsorbent and equilibrated for 1 hr at room temperature. The prepared solution was decanted completely. The resulting adsorbent was quick dried in previously maintained hot oven at 150° C. temperature for. 2 hrs. The resulting pore modified adsorbent was calcined at 250° C. under air flow for 4 hrs and named as modified 5A or PE 5A2. Prepared adsorbent PE5A and fresh ZMS 5A was characterized for inorganic silicate loading and adsorption uptakes for CO2 and ethylene were measured at 30° C. and 100-mmHg pressure. The prepared adsorbent contained 1.52% exchange of K+ ions, 70% Ca2+ and 26.5% of Na+ ions. Adsorption uptakes results show increase in fractional uptake rate of CO2 with respect to untreated adsorbent as shown in FIG. 1. 94% of total carbon dioxide adsorption capacity (after 60 minutes) could be achieved in first five minutes compared to 87% for fresh untreated adsorbent. Ethylene fractional uptakes remained constant after 5 minutes for PE 5A and untreated adsorbents as shown in FIG. 2 as 96% of total ethylene capacity (after 60 minutes) could be adsorbed. Diffusion time constants D/r² calculated from uptake data show faster diffusion of CO₂ for prepared adsorbent (6.66×10⁻⁴, D/r² sec⁻¹) compared to untreated adsorbent (5.12×10⁻⁴, D/r² sec⁻¹). Ethylene Diffusion time constants slightly decreased or remained constant compared to untreated molecular sieve ZMS A as given in Table 1. Water adsorption capacity measured on PE5A2 showed adsorption capacity of 20 wt % compared to 22 wt % unmodified ZMS A at 30° C. and 60RH as shown in Table 1. The prepared adsorbent was found suitable removal of hydrogen sulfide from ethylene gas. The prepared adsorbent adsorbed 15 wt % hydrogen sulfide at 30° C. with selectivity of 3 over ethylene.

### Example 2

Further gas mixture adsorption breakthrough's were measured in to estimate dynamic capacity at 30° C. and 10.5 Kg/cm2 (0.55% CO₂ balance ethylene) were measured on fresh ZMS CaA and modified ZMA CaA (PE 5A) apparatus as shown in FIG. 4. Feed gas mixture containing 0.5-0.6 wt % carbon dioxide gas was prepared by mixing CO₂ and ethylene in gas cylinder.

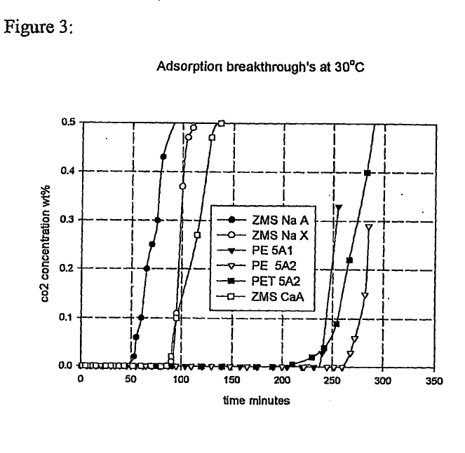

Adsorption breakthrough results on prepared adsorbent PE5A are shown and compared in FIG. 3. It can be seen that after pore modification there is substantial increase in breakthrough time of carbon dioxide and improvement in CO₂ adsorption capacity in the presence of ethylene. The details for adsorption breakthrough condition are given in table 2 for comparison. Breakthrough is defined as the point when the carbon dioxide concentration in the effluent rose from essentially zero to a detectable level of about 10 ppm. The pore modified ZMS PE2 showed the improved CO₂ adsorption capacity as 3.0 gm of CO₂/100 gm adsorbent could be adsorbed compared 1.4 gm of CO₂/100 gn of absorbent for unmodified Zeolite ZMS CaA molecular sieve. Similarly on ZMS NaA and NaX only 0.6 gm of CO₂ and 1.2 gm of CO₂/100 gm adsorbent could be adsorbed as can be seen in Table 2 and FIG. 3. It shows improvement in CO₂ adsorption capacity in the presence of ethylene after pore modification of ZMS A.

### Example 3

230 gm of the zeolite molecular sieve 5A, 1.5 mm extrudates after through mixing with 0.5 wt % metal silicate solution comprised of potassium prepared and characterized as per example 1 and named as PE5A1. Adsorption uptakes for CO₂ and Ethylene are shown in. FIGS. 1 and 2. The prepared adsorbent contained 0.95% exchange of K⁺ ions, 70% Ca²⁺ and 28.05% of Na⁺ ions. Adsorption uptake results show increase in fractional uptake rate of CO₂ with respect to untreated absorbent. 93% of total carbon dioxide adsorption capacity (after 60 minutes) could be achieved in first five minutes compared to 87% for fresh untreated adsorbent Ethylene fractional uptakes remained constant after 5 minutes on modified and untreated adsorbent as 96% of total ethylene capacity (after 60 minutes) could be adsorbed. Diffusion time constants D/r2 calculated from uptake data show faster diffusion of CO₂ for prepared adsorbent (6.04×10⁻⁴, D/r² sec⁻¹) compared to untreated (5.12×10⁻⁴, D/r² sec⁻¹). Ethylene Diffusion time constants slightly decreased or remained constant compared to untreated molecular sieve as given in Table 1. Water adsorption capacity measured on PE5A1 showed adsorption capacity of 20.5 wt % compared to 22 wt % unmodified ZMS CaA at 30 C and 60RH as shown in Table 1.

Adsorption breakthrough measured as example 2 on prepared adsorbent PE5A1 could adsorb 2.2 gm of CO₂/100 gm adsorbent compared to 1.4 gm of CO₂/100 gm of unmodified ZMS CaA adsorbent.

### Example 4

230 gm of the ZMS 5A, 1.5 mm extrudates after through mixing with 1.5 wt % metal silicate solution comprised of potassium prepared and characterized as per example 1 and named as PE5A3. The prepared adsorbent contained 1.95% exchange of K⁺ ions, 73% Ca²⁺ and 23.5% of Na⁺ ions. Adsorption uptakes results show increase in fractional uptake rate of CO₂ with respect to untreated adsorbent. 90% of total carbon dioxide adsorption capacity (after 60 minutes) could be achieved in first five minutes compared to 87% for fresh untreated adsorbent Ethylene fractional uptakes remained constant after 5 minutes for PE5A and untreated adsorbent as 96% of total ethylene capacity (after 60 minutes) could be adsorbed. Diffusion time constants D/r2 calculated from uptake data show faster diffusion of CO₂ for prepared adsorbent (5.42×10⁻³, D/r² sec⁻¹) compared to untreated adsorbent (5.12×10⁻⁴, D/r² sec⁻¹). Ethylene Diffusion time constants slightly decreased compared to untreated molecular sieve as given in Table 1. Water adsorption capacity measured on PE5A3 showed adsorption capacity of 19.5 wt % compared to 22 wt % unmodified ZMS at 30 C and 60RH as shown in Table 1.

Adsorption breakthrough measured as example 2 on prepared adsorbent PE5A3 could adsorb 1.56 gm of CO₂/100 gm adsorbent compared 1.4 gm of CO₂/100 gm of adsorbent for unmodified ZMS CaA adsorbent.

### Example 5

230 gm of the ZMS 5A, 1.5 mm extrudates after through mixing with 7.5 wt % metal silicate solution comprised of potassium prepared and characterized as per example 1. The prepared adsorbent contained 2.95% exchange of K⁺ ions, 79% Ca²+ and 17.5% of NA⁺ ions. Adsorption uptake results show increase in fractional uptake rate of CO₂ with respect to untreated adsorbent. 88% of total carbon dioxide adsorption capacity (after 60 minutes) could be achieved in first five minutes compared to 87% for fresh untreated absorbent Ethylene fractional uptakes remained constant after 5 minutes for PE5A and untreated adsorbent as 96% of total ethylene capacity (after 60 minutes) could be adsorbed. Diffusion time constants D/r2 calculated from uptake data show faster diffusion of CO₂ for prepared adsorbent (5.02×10⁻⁴, D/r² sec⁻¹) compared to untreated adsorbent (5.12×10⁻⁴, D/r² sec⁻¹). Ethylene Diffusion time constants slightly decreased compared to untreated molecular sieve as given in Table 1. Similarly water adsorption capacity measured on PE5A showed decrease adsorption capacity of 17.5 wt % compared to 22 wt % unmodified ZMS A at 30 C and 60RH as shown in Table 1.

Adsorption breakthrough measured as example 2 on prepared adsorbent PE5A adsorbed 1.0 gm of adsorbent for unmodified ZMS CaA adsorbent. Lower water and carbon dioxide adsorption capacity can be attributed to higher concentration of metal silicate solution resulting in low diffusional uptake of carbon dioxide.

### Example 6

adsorbent molecular sieve. Breakthrough is defined as the point when the carbon dioxide concentration in the effluent rose from essentially zero to a detectable level of about 10 ppm.

### Example 8

5 gm of 5A zeolite molecular sieve 1.5 mm extrudates were activated earlier at 250 C/4 hrs under nitrogen flow. 0.375 gm of Tetraethylorthosilicate (TEOS) was dissolved in 5 gm of toluene to prepare a TEOS solution and equilibrated for 1 hr at room temperature. The unadsorbed prepared TEOS solution was distilled off completely. The resulting adsorbent was dried and later oven dried at 100° C. temperature for 2 hrs. The resulting adsorbent was calcined at 510° C. under air flow for 5 hrs and named as TEOS Modified 5A or PET 5A1 in subsequent examples. Adsorbent was characterized for CO₂ uptakes as detailed in example-1. Results showed increase in fractional uptake rate of CO₂ with respect to untreated adsorbent as 93% of total carbon dioxide adsorption capacity (after 60 minutes) could be achieved in first five minutes compared to 87% for fresh untreated adsorbent. Diffusion time constants D/r2 calculated from uptake data show faster diffusion of CO₂ for prepared adsorbent (8.31×10⁻⁴, D/r² see) compared to untreated adsorbent (5.12×10⁻⁴, D/r² sec⁻¹). Ethylene Diffusion time constants remained almost constant compared to untreated molecular sieve as given in Table 1.

### References

1. “Regenerable solid amine sorbent”, Birabara Philip J., Filburn; Thomas P. and Nalette Timothy A. U.S. Pat. No. 5,876,488.
2. “CO2 removal from gaseous streams”, Slaugh; Lynn H. and Willis; Carl L. U.S. Pat. No. 4,433,981.
3. “Process for regenerative sorption of CO2” Dimitri Gidaspow and Michael Onischak, U.S. Pat. No. 3,865,924.
4. “Molecular sieve adsorbent for gas purification thereof” Rode; Edward J. and Tsybulevskiy; Albert M. U.S. Pat. No. 6,530,975.
5. “Molecular sieve adsorbents” Robert M Milton, U.S. Pat. No. 2,882,244.
6. “Carbon dioxide removal from vapour mixtures” Robert M Milton, U.S. Pat. No. 3,078,639.
7. “Gas Purification” Arthur Kohl and Richard Nielson, 1997, 5^{th} Edition, chapter 12 “Gas Dehydration and Purification by Adsorption” page number 1076. Gulf Publishing Co. Houston.

TABLE 1

| | | | | | WAC |
|---|---|---|---|---|---|
| | CO2, D/r², | Ethylene, D/ | CO2 ads. | C2H4 ads. | 60RH |
| Adsorbent | sec⁻¹ | r², sec⁻¹ | wt % | Wt % | wt % |
| ZMS 5A | 5.12E−04 | 7.24E−04 | 15.11 | 7.31 | 22.0 |
| PE 5A 1 | 6.04E−04 | 7.04E−04 | 12.70 | 5.61 | 20.5 |
| PE 5A 2 | 6.66E−04 | 7.03E−04 | 13.18 | 5.68 | 20.0 |
| PE 5A 3 | 5.42E−04 | 6.23E−04 | 12.06 | 4.75 | 19.5 |
| PE 5A | 5.02E−04 | 6.01E−04 | 11.66 | 3.95 | 17.5 |
| PET 5A | 6.96E−04 | 7.23E−04 | 12.64 | 5.99 | 20.8 |
| PET 5A1 | 8.31E−04 | 7.23E−04 | 14.08 | 5.98 | 20.5 |
| PET 5A2 | 9.55E−04 | 7.52E−04 | 17.65 | 7.72 | 21.7 |

TABLE 2

| | | | | Gm of CO2 |
|---|---|---|---|---|
| | | | | adsorbed/100 |
| | Adsorbent | Feed rate | CO2 concentration in | gm of |
| Adsorbent | wt, g | Ml/min | ethylene feed, wt % | adsorbent |
| MS NaA | 198 | 2150 | 0.6 | 0.6 |
| ZMS CaA | 190 | 2350 | 0.65 | 1.4 |
| ZMS NaX | 188 | 2300 | 0.57 | 1.2 |
| PE 5A1 | 193 | 2300 | 0.55 | 2.2 |
| PE 5A2 | 190 | 2350 | 0.55 | 3.0 |
| PET 5A2 | 185 | 2200 | 0.6 | 2.7 |

We claim:

1. An adsorbent for removing impurities such as CO₂, H₂S and water vapors from a gaseous olefin stream of at least C₂ to C₄ olefins, which comprise zeolite CaA molecular sieve modified with metal silicate.

2. An adsorbent as claimed in claim 1 wherein said metal silicates are selected from organic silicates and inorganic metal silicates.

3. An adsorbent as claimed in claim 2 wherein said inorganic metal silicates are selected from silicates of potassium, sodium or mixture thereof.

4. An adsorbent as claimed in claim 2 wherein said organic silicates are selected from tetraethyl orthosilicate, tetrapropyl orthosilicate or mixture thereof.

5. An adsorbent as claimed in any preceding claim wherein said zeolite CaA comprises zeolite 5A molecular sieve modified with metal silicate in the concentration range of 0.5% to 10%.

6. A process for the preparation of an adsorbent for use in removing impurities such as CO₂, H₂S and water vapors from a gaseous olefin stream of at least C₂ to C₄ olefins which comprises treating a calcium form of Zeolite A with a solution of silicate (Zeolite CaA), drying said treated Zeolite CaA and calcining said dried silicate at a temperature in the range of 100-600° C. to obtain said adsorbent.

7. A process as claimed in claim 6, wherein said calcination is carried out for a period of from 0.1 to 100 hrs.

8. A process as claimed in claim 7 wherein said calcination is carried out for 1 to 10 hours.

9. A process as claimed in claim 7 or 8 wherein said solution of silicates comprises of inorganic silicate dissolved in a suitable solvent like water in concentration range of 1 to 20%.

10. A process as claimed in any one of claims 7 to 9 wherein said calcium form of zeolite A is treated with 0.1 wt % to 15 wt % of a solution of silicate solution in the range of 0.1 wt % to 15 wt % and equilibrated for a period or 0.1 to 24 hrs preferably, for 1 to 2 hrs.

11. A process as claimed in any one of claims 7 to 9 wherein said calcination is carried out in a suitable atmosphere such as nitrogen and helium.

12. A method for removing impurities such as CO₂, H₂S and water vapors from a gaseous olefin stream of at least C₂ to C₄ olefins which comprise passing said gaseous olefin stream containing said impurities into contact with an adsorbent comprising of zeolite CaA molecular sieve modified with metal silicate.

13. A method as claimed in claim 12 wherein said inorganic metal silicates are selected from silicates of potassium, sodium or mixture thereof.

14. A method as claimed in claim 12 wherein said organic silicates are selected from tetraethyl orthosilicate, tetrapropyl orthosilicate or mixture thereof.

15. A method as claimed in any one of claims 12 to 14 wherein said zeolite CaA comprises zeolite 5A molecular sieve modified with metal silicate in the concentration range of 0.5% to 10%.

16. A method as claimed in any one claims 12 to 15 wherein said olefin feed stream comprises of ethylene containing 0.01% to 1% carbon dioxide along with trace amount of methane, ethane and oxygen further containing 0.01% to 0.5% of carbon dioxide.

17. A method as claimed in any one of claims 12 to 16 wherein said adsorbent is in the form of a particulate bed.

18. A method as claimed in any one of claims 12 to 18 wherein the adsorbent bed temperature is in the range of 10 to 120° C. and preferably, 30 to 60° C.

19. A method as claimed in any one of claims 12 to 19 wherein olefin feed stream temperature is in the range of 20-80° C. and preferably, 30 to 60° C.

20. A method as claimed in any one of claims 12 to 20 wherein olefin feed stream pressure is in the range of 2 to 20 kg/cm², preferably, 10 to 15 kg/cm².

21. A process as claimed in claim 12 wherein said impurity is H₂S or water, said olefin is ethylene or propylene and the temperature is in range of 10-80° C., preferably, 30-50° C.
